(19) United States
(12) Patent Application Publication
(10) Pub. No.: US 2008/0124433 A1
(43) Pub. Date: May 29, 2008

(54) Food processing using laser radiation

(75) Inventors: Eugene F. Yelden, Mukilteo, WA (US); David Clarke, Seattle, WA (US)

Correspondence Address: DARBY & DARBY P.C., P.O. BOX 770, Church Street Station, New York, NY, 10008-0770, US

(73) Assignee: Synrad, Inc., Mukilteo, WA (US)

(21) Appl. No.: 11/563,619

(22) Filed: Nov. 27, 2006

(51) Int. Cl. A23L 1/025; B23K 26/38; B23K 26/02; G01N 33/02

(52) U.S. Cl. 426/233; 219/121.61; 219/121.62; 219/121.7; 219/121.74; 426/240

(57) ABSTRACT

Processing food with a laser. In one embodiment, the system includes a laser that creates holes in a food item with an output beam, and includes a post processing module that enables a substance to pass through the holes. The substance may include a flavoring, a vitamin, a medicine, a gas, steam, and the like. The holes facilitate cooking, cleaning, flavorizing, medicating, off-gassing, eating, or the like. The laser system may also perforate or score a food item shell to ease cracking and access to interior material. The system may also include one or more mirrors, focus heads, delivery optics, beam scanners, or the like, to control output beam characteristics, such as focus and positioning. In another embodiment, a dynamic control system may add computerized-feedback and control to the laser system. A position system may also control a position, orientation, or other characteristic of a food target.

### FIELD OF ART

The present invention relates generally to food processing and, more particularly, but not exclusively, to enabling laser assisted generation of small holes in food.

### BACKGROUND

Lasers are becoming more ubiquitous in a wide range of varied applications. It is now commonplace to encounter some form of laser in supermarket scanners, CD/DVD players or even in leveling tools used to aid in picture/shelf hanging. Products that have been, to one extent or another, influenced, processed or enhanced by the use of laser radiation also frequently surround us. For instance, product marking and packaging are but two example areas of laser use. This use may involve simple package marking with date-code and UPC-type markings for inventory and stock control. Another application is EggFusion marking two-dimensional barcodes on eggs for identification and tracking purposes. Similarly, date and lot-code markings on plastic bottles are applied with CO₂ lasers as an environmentally cleaner, simpler and, arguably, less-expensive, alternative to ink jet marking systems. Further examples of the use of lasers in the food industry include the marking of cheeses for lot, batch and quality control, the marking of breads and meats for similar purposes and even the marking, or ‘tattooing’ of various fruits (limes, lemons, oranges, etc.) as a means of tracking, identification, promotion, and advertising.

Other examples of the use of lasers in food-related industries include the area of controlled atmospheric packaging, which is perhaps more commonly referred to as MAP (modified atmospheric packaging). MAP involves a means of altering and controlling the rate of diffusion of oxygen, carbon dioxide, and nitrogen (plus other gases) into and out of a fresh produce container, by varying the size, location, and number of holes, in an effort to extend the freshness and shelf life of the produce. Therefore, it is with respect to these considerations and others that the present invention has been made.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a system diagram illustrating a laser system according to one embodiment of the invention;

FIG. 2 is a system diagram illustrating a food processing system according to one embodiment of the invention;

FIG. 3 shows one embodiment of the invention for a multi-sided exposure; and

FIG. 4 is a logical flow diagram illustrating logic flow operations according to one embodiment of an overview of a process to score holes in a target.

### DETAILED DESCRIPTION

The present invention now will be described more fully hereinafter with reference to the accompanying drawings, which form a part hereof, and which show, by way of illustration, specific embodiments by which the invention may be practiced. This invention may, however, be embodied in many different forms and should not be construed as limited to the embodiments set forth herein; rather, these embodiments are provided so that this disclosure will be thorough and complete, and will fully convey the scope of the invention to those skilled in the art. Among other things, the present invention may be embodied as methods or devices. The following detailed description is, therefore, not to be taken in a limiting sense.

Throughout the specification and claims, the following terms take the meanings explicitly associated herein, unless the context clearly dictates otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

In addition, as used herein, the term “or” is an inclusive “or” operator, and is equivalent to the term “and/or,” unless the context clearly dictates otherwise. The term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.”

Briefly stated the present invention is directed to food processing that may be enhanced through the application of laser irradiation and, in particular, the formation of one or more holes into at least the outer layer of the food item. The invention is also directed to any process that involves the diffusion of material and/or energy, such as water, heat, steam, flavor, vitamins, dietary supplements, medicines, gases, and the like into or out of the food item. For example, the addition of flavor into a food item is often referred to as an ‘infusion’ process and can also include sugars, syrups, liquids, or the like, as well as gasses, vapors, or the like.

In one embodiment, the time required to process a food item may be reduced, reducing the energy required, improving the yield, taste, texture of the product or some combination of the above. However, the invention is not limited to this embodiment. For example, other process parameters or properties may be affected by laser irradiation.

### Illustrative Operating Environment

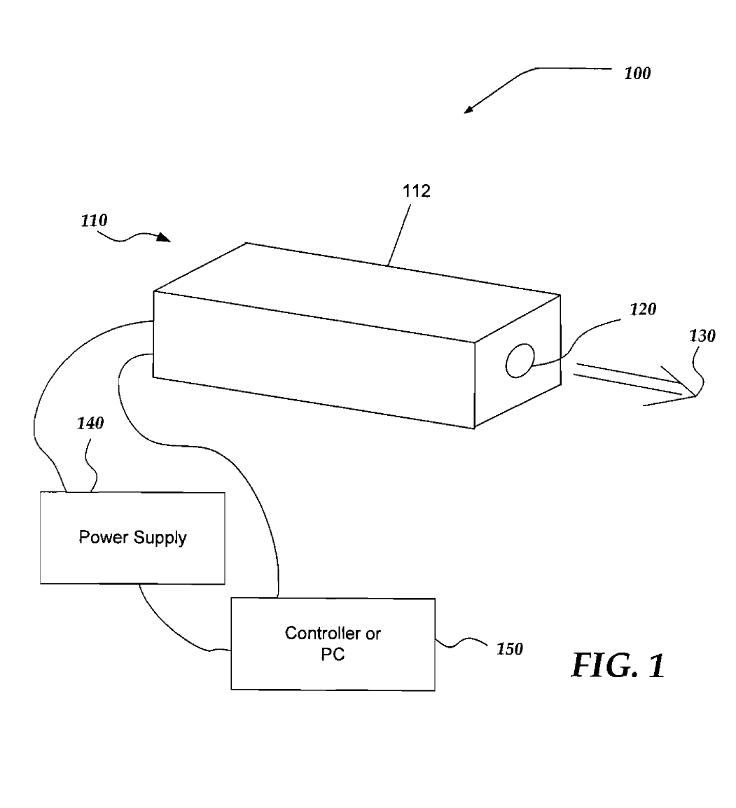

FIG. 1 shows a laser system 100 according to one embodiment of the invention. Not all the components may be required to practice the invention, and variations in the arrangement and type of the components may be made without departing from the spirit or scope of the invention. As shown, laser system 100 of FIG. 1 includes a laser 110 with an optional sealed case 112, an output aperture 120, a power supply 140 and a controller 150.

Generally, controller 150 may include virtually any control device capable of controlling the operation of laser 110. Examples include, but are not limited to, personal computers, laptops, or the like. Laser 110 is configured to couple with power supply 140 and controller 150. Controller 150 may also be optionally coupled directly or indirectly to power supply 140. Power supply 140 provides power to laser 110. A rechargeable or non-rechargeable battery may be used to provide power. The power may also be provided by an external power source, such as a DC power supply or an AC adapter that supplements and/or recharges a battery. Laser 110 may be optionally sealed in an enclosure 112, which includes an output aperture for an irradiating output beam 130.

### Illustrative Output Beam Environment

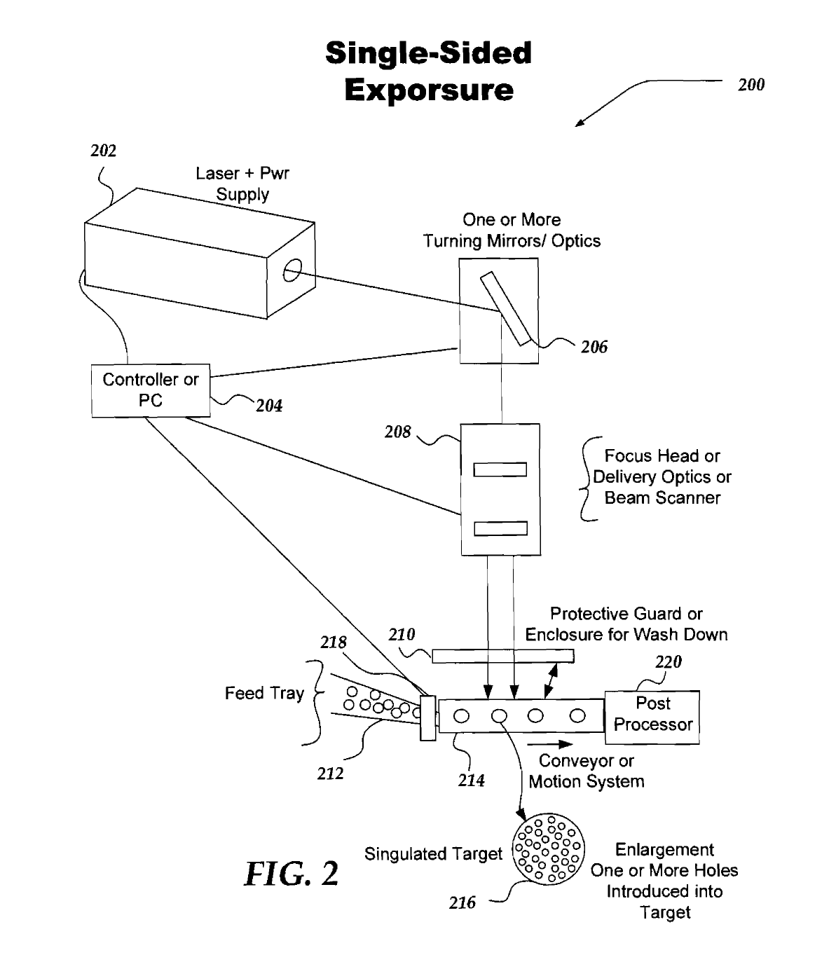

FIG. 2 shows a food processing system 200 according to one embodiment of the invention. In this embodiment, food is subject to a single-sided exposure from laser irradiation. This single-sided exposure laser food processing system 200 may include many more or less components than those shown in FIG. 2. However, the components shown are sufficient to disclose an illustrative embodiment for practicing the present invention.

As shown in the figure, the single-sided exposure laser food processing system 200 includes a combined laser and power supply 202, a controller 204, one or more turning mirrors or optics 206, a focus head 208, a protective guard 210, a feed tray 212, a position system 214, and a post processor 220. Laser irradiation is directed toward a target 216, such as a fruit or other food item.

Controller 204 may be optionally combined with a sensor 218 designed to detect information regarding the target, such as surface or size irregularities, or the like. Sensor 218, other sensors, and/or controller 204 detect one or more characteristics of the laser, the output aperture, an output beam, and/or a food target. The sensors generally provide information to the controller, including a position of the food target, a condition of at least a portion of the food item, a path of the output beam, an intensity of the output beam, a focal point of the output beam, a duration of the output beam, and/or other characteristics. Controller 204 in conjunction with the optics 206 and/or focus head 208 may dynamically adjust the focus, direction, power level, size, intensity, or the like of the laser output beam. Controller 204 and/or a controller near sensor 218 may cause actuators and/or other devices to modify a position, orientation, speed, or other aspect of the food target.

Turning mirror 206 may optionally direct the output beam. There may be zero, one or more turning mirrors or optics 206 to direct the output beam. In one embodiment, the output beam may then pass through a focus head 208 to focus the output beam. However, the invention is not limited to this focus head. For example, the output beam may optionally pass through delivery optics and/or a beam scanner in conjunction with, or in place of, the focus head 208. Processing on the output beam may include zero, one or more of these various options, combined in any configuration.

Protective guard 210 may provide a barrier between the beam processing mechanism and the position system 214. The position system 214, such as a conveyor, may transport targets from a feed tray 212 across the site of the output beam. Feed tray 212 provides a means to singulate the individual food item so that each individual piece may pass under or through the laser beam. However, the invention is not limited by this embodiment. For example, the output beam may be scanned across a line, an area, or the like, by using galvanometer steering optics. The output beam may drill holes into the target 216. Irradiation may put at least one, but possibly several, relatively small or microscopic holes in the target 216. Example hole diameters may include approximately 50-250 μm. Hole diameters and depths may depend on characteristics of the target.

One example target is rice grains. Rice essentially cooks from one end of the grain. The required cooking time is generally the length of time for water/heat/steam to effectively diffuse from one end of the grain to the other end. By introducing one or more small holes along the length of the grain the cooking time may be reduced, because the effective diffusion length, and hence the time to diffuse throughout the entire grain, is significantly reduced. According to a diffusion equation, the time required for diffusion over a given distance depends directly on the length of that distance. Hence, a shorter distance generally equates to a shorter time requirement. The invention is not limited by this example. Other examples may include other food items as well. For example, beans (e.g., kidney, flava, or the like) would also cook well with this type of processing.

In general, the addition of numerous small holes into the target via laser irradiation may reduce the time for diffusion or infusion into or out of the target. In the case of cooking, for example (but not limited to), precooking by parboiling, may reduce the time required to cook the food. This time reduction is generally based on, and related to, the diffusion of heat and steam into the entire food product. Therefore, by introducing small holes into the surface of the food, the characteristic diffusion length to completely cook the item may be significantly reduced. Cooking is one example of post processing performed by post processor 220. Other examples include infusing a substance through the holes into the food target. The substance may be a flavoring, a medicine, a gas, or other material. Conversely, post processor 220 may apply pressure, heat, a catalyst, or other process on the food target to cause gas, oil, soft core material, or other substances to pass out through the holes.

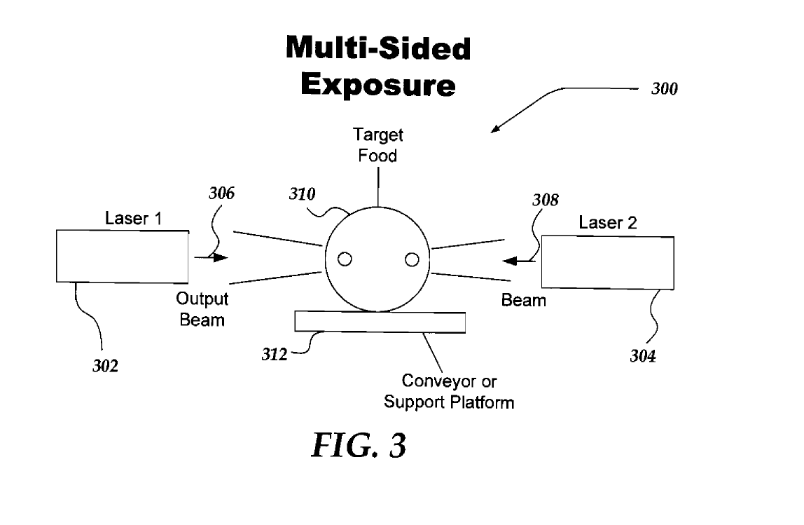

FIG. 3 shows one embodiment of the invention for multi-sided exposure. A multi-sided exposure laser system 300 may include many more or less components than those shown in FIG. 3. However, the components shown are sufficient to disclose an illustrative embodiment for practicing the present invention.

The multi-sided exposure laser system 300 is shown in a simplified configuration to include only lasers 302 and 304, output beams 306 and 308, and position system or platform 312. Output beams 306 and 308 are directed to a target 310 from differing directions. However, multi-sided exposure laser systems are not limited by this embodiment. For example, system 300 may include more or less components than those shown in FIG. 2, such as one or more sensors, controllers, and a feed tray.

The position system 312, shown in FIG. 3, may include, for example, a conveyor, a stationary support platform, a channel, a clamp, or the like. The position system generally holds the target 310 in place so that holes can be created in the target by output beams 306 and 308. Position system 312 may also rotate, translate, tilt, raise, lower, or otherwise reposition target 310.

In one embodiment, the holes in the target may be used for an infusion process for sweetening or for flavoring foods. This process benefits from laser irradiation treatment of the food prior to completion of the process. Many foods, such as fruits (blueberries, cranberries, cherries, grapes, or the like) or vegetables have a relatively hard and impenetrable outer layer of skin. This skin forms a protective layer for the typically much softer, inner flesh of the product. However, this outer layer of skin, may also be an effective barrier against the infusion of sugar, syrup, flavor, vitamins, pharmaceuticals, or the like into the food item. An adequate process either takes a substantial amount of time or some mechanical means of perforating the outer layer. This process is often called maceration and involves the use of several blades or knives to cut the outer, protective layer of the food. Examples of this type of processed food product are a glacee blueberry, a maraschino cherry, or the like. The act of physically cutting/penetrating the skin with a mechanical device may degrade the physical appearance to the item. The mechanical device used to achieve this penetration may also be subject to wear and deterioration, thereby eventually requiring replacement. Plus, there may be food contamination risk due to the blades (or needles, or the like) coming in contact with each item. Irradiation using an appropriate laser source may overcome these drawbacks with the additional benefit of reducing the time to achieve an adequate level of flavor, sweetness, medicinal infusion, or other saturation. The flavorant or other infusion material can penetrate directly into the inner flesh/meat of the food item through the laser-created holes.

### Generalized Operation

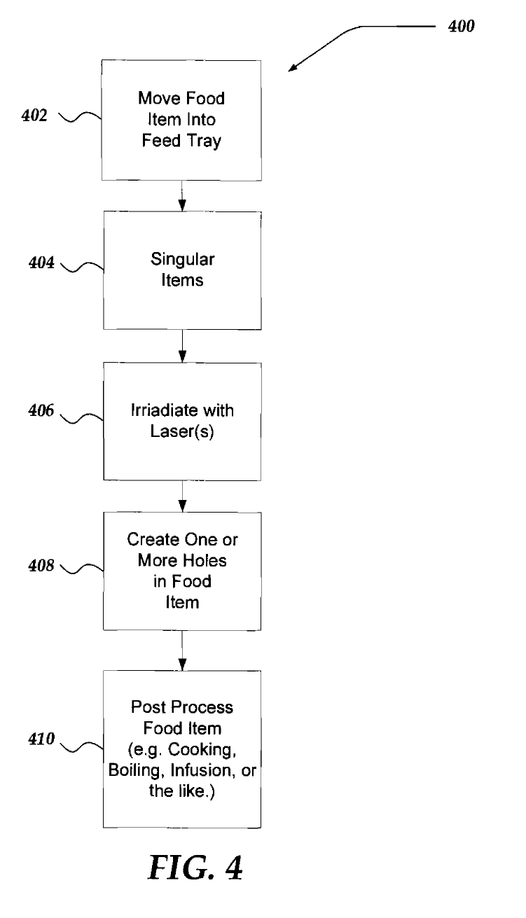

The operation of certain aspects of the invention will now be described with respect to FIG. 4. FIG. 4 illustrates a logical flow diagram generally showing one embodiment of an overview of process 400 for generating holes in food.

As shown, process 400 begins, at block 402, wherein one or more food items may be placed into a feed tray. The food items may then be separated into singular items at block 404. Next, the singular food items may be transported on a position system across the path of one or more lasers' output beams. Block 406 energizes the laser(s) to irradiate the food items with the laser output beam(s). At block 408, the laser output beam(s) create one or more holes in each food item. Finally, at block 410, the food items are ready for continued food processing, such as cooking, boiling, infusion, or the like.

In one embodiment, certain foods may be infused with flavor without using alcohol that often leaves behind a residual alcohol component. The flavoring process may be enhanced by using a laser treatment, such as puncturing with holes, slits, or the like, to enable more effective penetration of a flavor component into the food without using an alcohol component. In another embodiment, a laser removes or evaporates any residual alcohol component from the food item during the flavoring process. This may be accomplished by selecting or setting the laser to dissociate or break the molecular bonds of the alcohol while leaving the flavor molecules intact.

In another embodiment, the process of freeze-drying includes the removal of water and liquids from a fruit, vegetable, or the like. This is typically facilitated by macerating the outer skin of the produce (often by knife blade, pin, needle, or the like), to more readily allow the water to be removed. This process can damage the food item. However, radiation by a laser overcomes this drawback by introducing one or more holes that penetrate through the outer, protective layer to allow the moisture to be rapidly evacuated from the target. These holes are large enough to allow water molecules to escape and multiple, uniformly spaced holes allow for faster and more uniform processing of the target. This process generally works with any type of frozen, freeze-dried and/or dehydrated vegetable or fruit, such as carrots, corn, beans, peas, apples, peaches, plums, pears, cherries, cranberries, or the like, to give but a few examples.

In another embodiment, laser holes are used to enhance processing for French fries. The French fry industry produces millions of pounds of frozen potato products each day. Laser holes aid in pre-cooking or parboiling prior to coating and preparing the fry for the deep fryer. Holes also improve diffusion of water/steam/heat into the fry and accelerate the initial cooking that is generally performed prior to a final frying stage.

In another embodiment, laser holes modify the texture and mouth-feel of the food. The texture relates to how the item is perceived once it is in the mouth of a consumer. Changing the surface by a laser can enhance or otherwise change the texture of a food, making it more palatable.

In another embodiment, coffee processing is performed with the above-mentioned processes, such as during the decaffeination process. Additionally, coffee releases gasses after the roasting process. Due to this gas release, the coffee generally may not be packaged until it has reached equilibrium (e.g., up to 24 hrs) or the packaging employ some additional feature, such as a one-way pressure relief valve, to allow the gas to escape without causing the package to burst or explode. However, treating the roasted beans with a laser by introducing one or more small holes allows the gas to escape from the roasted beans faster and reduces or eliminates the need for a relief valve in the package.

In another embodiment, laser treatment of food products enhances, or enables, creation of nutritionally enhanced or fortified foods and/or the possibility of functional and nutraceutical foods. For example, some foods lose their nutritional value at various steps during the processing chain due to the nature of the processing. Alternatively, various additives may be put into certain foods to enhance their functionality, nutritional provision, or even medicinal effectiveness. Using a laser to put holes into various foods enhances or enables this process to occur more effectively than it can currently be carried out.

In another embodiment, lasers are used to mark and/or score shellfish, crustaceans, nuts, or the like in a manner that facilitates removal of an interior material, such as meat and flesh from inside food target. For example, scoring a line or a sequence of perforation holes down or along a crab or lobster claw with a CO₂ laser reduces the effort required to open the claw and retrieve the meat inside. This is more efficient and less damaging to the flesh than using a mechanical device to crush or break apart the shellfish.

In another embodiment, the lasers are employed to mark the outer shells to identify such qualities as brand, location, age, time of harvest, or the like.

It will be understood that each block of the flowchart illustrated in FIG. 4, and combinations of blocks in the flowchart illustration, can be implemented by computer program instructions. These program instructions may be provided to a processor to produce a machine, such that the instructions, which execute on the processor, create means for implementing the actions specified in the flowchart block or blocks. The computer program instructions may be executed by a processor to cause a series of operational steps to be performed by the processor to produce a computer implemented process such that the instructions, which execute on the processor to provide steps for implementing the actions specified in the flowchart block or blocks. The computer program instructions may also cause at least some of the operational steps shown in the blocks of the flowchart to be performed in parallel. Moreover, some of the steps may also be performed across more than one processor, such as might arise in a multi-processor computer system. In addition, one or more blocks or combinations of blocks in the flowchart illustration may also be performed concurrently with other blocks or combinations of blocks, or even in a different sequence than illustrated without departing from the scope or spirit of the invention.

Accordingly, blocks of the flowchart illustration support combinations of means for performing the specified actions, combinations of steps for performing the specified actions and program instruction means for performing the specified actions. It will also be understood that each block of the flowchart illustration, and combinations of blocks in the flowchart illustration, can be implemented by special purpose hardware-based systems which perform the specified actions or steps, or combinations of special purpose hardware and computer instructions.

### Sample Operation

Embodiments of the present invention were implemented and tested. A sample of such embodiments and tests are described below. In one such test, a sample of approximately 2 kg of blueberries was divided into three equal portions of 680 grams each. One portion was retained as a control group and the other two portions of berries were designated to be irradiated using a CO₂ laser with output radiation at 10.6 μm. An 80 W CO₂ laser connected to a galvanometer based marking head with a 200 mm focal length lens was used to process the berries. This lens produced approximately 290 μm diameter holes (1/e² width) in the berries with a 5 mm depth of focus, The laser was set to a power level of 60 W with a 1 msec pulse width. Holes were created in the berries in an array pattern with a hole spacing of either 2 mm or 4 mm, for the remaining two groups. Each berry had at least one hole created through its outer skin/protective layer, although many had several more holes.

After treatment with the laser, the berries were placed into a corn syrup solution to begin an infusion process. This process usually takes several days and is typically considered to have successfully produced glacee fruit when the Brix value of the fruit reaches a value of between 70-74. Brix value is a measure of the sugar percentage content. As a reference, pure corn syrup has a Brix value of 78.5. As detailed above, the blueberries were divided into three groups. The berries were left in the corn syrup solution for several days (the pure syrup solution was topped up as required) and measurements of their Brix value were taken daily. After a period of eight days, the non-laser treated control group of fruit obtained a Brix level of 65.62, which typically is not an adequate value to be considered glacee fruit. However, in the same time period, both of the laser treated samples had achieved higher Brix values (71.1 for the 2 mm hole spacing and 70.35 for the 4 mm hole spacing). The laser treated blueberries are typically considered to be glacee fruit. Additionally, from a qualitative perspective, the laser treated berries had a better physical appearance than the non-treated fruit in that the berries were plumper and exhibited less shriveling than the control group.

The examples provided should not be construed as narrowing the embodiments of the invention, and are intended merely to provide a better understanding. Thus, other mechanisms may therefore be employed, without departing from the scope of the invention.

The above specification, examples, and data provide a complete description of the manufacture and use of the composition of the invention. Since many embodiments of the invention can be made without departing from the spirit and scope of the invention, the invention resides in the claims hereinafter appended.

What is claimed as new and desired to be protected by Letters Patent is:

1. A method for processing a food item, comprising:
creating a hole in the food item with an output beam of an irradiation laser; and
causing a substance to pass through the hole, wherein passing the substance changes a characteristic of the food item.

2. The method of claim 1, wherein the hole has a diameter in the range of 50-300 μm.

3. The method of claim 1, further comprising:
modifying the path of the output beam using at least one turning mirror.

4. The method of claim 1, further comprising modifying the output beam using at least one of the following: a focus head, a delivery optic, and a beam scanner.

5. The method of claim 1, wherein the substance comprises at least one of the following: a vitamin, a dietary supplement, a flavor, a gas, a vapor, and a medicine.

6. The method of claim 1, further comprising at least one of the following:
separating the food item from a plurality of other food items prior to creating the hole; and
conveying the food item along a position system, such that the food item travels across a path of the output beam.

7. The method of claim 1, further comprising creating at least one additional hole in the food item by one of the following:
moving a path of the output beam; and
energizing at least one additional irradiation laser that applies at least one additional output beam to the food item.

8. The method of claim 1, further comprising causing trapped gas within the food item to pass through the hole.

9. The method for processing food in claim 1, further comprising:
monitoring the food item with a sensor;
providing a feedback to a controller from the sensor; and
modifying at least one of the following: a focus, a power level, a beam size, and a position of the output beam, based on the feedback.

10. The method for processing food in claim 1, further comprising:
marking the food item with the irradiation laser to identify the food item.

11. An apparatus, comprising:
a power supply;
an irradiation laser coupled to the power supply and configured to create a hole in a food item with an output beam of the irradiation laser; and
a post processor causing a substance to pass through the hole, wherein passing the substance changes a characteristic of the food item.

12. The apparatus of claim 11, further comprising a mirror located in a path of the output beam to modify a direction of the output beam.

13. The apparatus of claim 11, further comprising an optic located in a path of the output beam to modify the output beam.

14. The apparatus of claim 11, wherein the substance comprises at least one of the following: a vitamin, a dietary supplement, a flavor, a gas, a vapor, and a medicine.

15. The apparatus of claim 11, further comprising at least one of the following:
a feed tray configured to separate the food item from a plurality of other food items; and
a position system configured to position the food item in a path of the output beam.

16. The apparatus of claim 11, further comprising:
a controller; and
a position system coupled to the controller and modifying a position of the food item in response to an instruction from the controller.

17. The apparatus of claim 11, further comprising a controller coupled to the power supply and to the irradiation laser; wherein the controller modifies a characteristic of the output beam.

18. The apparatus of claim 17, further comprising a sensor coupled to the controller and providing information to the controller, including at least one of the following: a position of the food item, a condition of at least a portion of the food item, a path of the output beam, an intensity of the output beam, a focal point of the output beam, a power level of the output beam, a size of the output beam, and a duration of the output beam.

19. The apparatus of claim 11, further comprising at least one additional irradiation laser coupled to the power supply and configured to create at least one additional hole in the food item with at least one additional output beam of the at least one additional irradiation laser.

20. A method for processing a food item, comprising:
applying an output beam of an irradiation laser to a surface of the food item to create a crack location that enables the surface of the food item to be opened at the crack location.

21. The method of claim 20, wherein the food item comprises one of the following, a shellfish, a crustacean, and a nut.

22. The method of claim 20, wherein the crack location comprises a sequence of perforation holes in the surface of the food item.

23. The method of claim 20, wherein the crack location enables access to an interior material of the food item.

24. An apparatus for processing a food item, comprising:
a power supply;
an irradiation laser coupled to the power supply and configured to produce an output beam; and
an optic positioned in a path of the output beam and modifying the output beam to enable the output beam to produce a hole in the food item.

25. The apparatus of claim 24, wherein the optic comprises one of the following: a mirror, a lens, and a scanner.
